(19) United States
(12) Patent Application Publication
(10) Pub. No.: US 2023/0157575 A1
(43) Pub. Date: May 25, 2023

(54) SYSTEMS FOR EVALUATING RESPIRATORY FUNCTION USING FORCED OSCILLATION TECHNIQUE (FOT) OSCILLOMETRY

(71) Applicant: Respiratory Sciences, Inc., Minneapolis, MN (US)

(72) Inventors: Jesse J. Cole, Plymouth, MN (US); Patrick A. Lichter, Minneapolis, MN (US); Robert J. Knuesel, Minneapolis, MN (US); Andrew T. Alexander, Murrysville, PA (US)

(21) Appl. No.: 18/058,702

(22) Filed: Nov. 23, 2022

Related U.S. Application Data
(60) Provisional application No. 63/282,409, filed on Nov. 23, 2021.

(51) Int. Cl. A61B 5/085; A61B 5/087; A61M 16/00

(52) U.S. Cl. CPC A61B 5/085; A61B 5/087; A61M 16/0003; A61M 16/024; A61M 2016/0027

(57) ABSTRACT

Systems for evaluating the respiratory function of an individual using forced oscillation technique (FOT) oscillometry include a blower controlled so as to apply FOT pressure oscillations on top of a low amplitude offset pressure. A controller continually adjusts the rotational speed of the blower to maintain a targeted time-varying pressure profile in the breathing air provided to the patient.

### CROSS-REFERENCE TO RELATED APPLICATIONS

This application claims the benefit under 35 U.S.C. 119(e) of U.S. provisional application No. 63/282,409, filed Nov. 23, 2021, the contents of which are incorporated by reference herein in its entirety.

### BACKGROUND

Forced oscillation technique (FOT) oscillometry is a method of respiratory oscillometry for measuring the mechanical properties of the lungs and airways. FOT often is used to diagnose asthma, COPD, and other respiratory ailments. FOT involves superimposing a stimulus, in the form of a sinusoidally-varying pressure waveform, over the normal tidal breathing of a patient. The response of the patient's respiratory system to the FOT pressure waveform is determined by measuring the resulting changes in air flow and the pressure waveform as the patient is tidal breathing. Based on these measurements, the impedance of the respiratory system can be calculated, which in turn can assist physicians and other clinicians in evaluating lung function and diagnosing respiratory ailments.

The FOT pressure waveform typically is a sinusoidally-varying pressure fluctuation of low-frequency, and low amplitude. For example, typical FOT pressure waveforms can have a frequency of about 5 Hz to about 50 Hz, and a peak-to-peak amplitude of about 0.1 cm H₂O to about 2 cm H₂O. Pressure waveforms with these characteristics are difficult to produce using rotating machinery such as blowers, due to difficulties in controlling the rotating mass of the blower with the accuracy and stability needed to reliably produce such low-frequency, low-amplitude pressure fluctuations. Thus, systems and devices used to perform FOT diagnostics typically employ a speaker or a vibrating mesh to produce the FOT pressure waveform.

### SUMMARY

In one aspect of the disclosed technology, a system for evaluating the respiratory function of an individual using forced oscillation technique oscillometry includes a blower having a casing, an impeller mounted for rotation within the casing, and a motor configured to, during operation, rotate the impeller. The system also incudes a ventilation interface, and a connecting member defining a passageway in fluid communication with the blower and the ventilation interface.

The system further incudes a control unit communicatively coupled to the motor and configured to control a rotational speed of the impeller to meet a set of rotational speed setpoints for the impeller so that the blower produces a time-varying pressure waveform in the passageway, the time-varying pressure waveform including a sinusoidally-varying pressure fluctuation to be superimposed on a respiratory flow of the patient by way of the ventilation interface, and an offset pressure selected to maintain a positive air pressure in the passageway during operation of the system.

In another aspect of the disclosed technology, the control unit is further configured to control the rotational speed of the impeller to meet the set of rotational speed setpoints by generating a control input based on a desired air pressure with the passageway, and a known relationship between the rotational speed of the impeller and an air pressure produced by the blower. The motor is configured so that the motor varies the rotational speed of the impeller in response to the control input.

In another aspect of the disclosed technology, the control input is a single-frequency signal.

In another aspect of the disclosed technology, the control input is a multi-frequency signal.

In another aspect of the disclosed technology, the controller is further configured to generate the control input by combining at least a first and a second signal.

In another aspect of the disclosed technology, an amplitude, a phase, and a waveform of the first signal are different than a respective amplitude, phase, and waveform of the second signal.

In another aspect of the disclosed technology, the offset pressure is about 0.5 cm H₂O to about 40 cm H₂O.

In another aspect of the disclosed technology, the offset pressure is substantially constant. In another aspect of the disclosed technology, a maximum pressure amplitude of the time varying pressure waveform is about 0.1 cm H₂O to about 2 cm H₂O.

In another aspect of the disclosed technology, the system further includes an outlet port in fluid communication with the passageway in the connecting member, and an ambient environment around the system.

In another aspect of the disclosed technology, the outlet port has a length of about zero to about three inches.

In another aspect of the disclosed technology, the passageway and the outlet port form an airflow pathway between the ventilation interface and the ambient environment around the system; and the system the further includes an obstruction located within the outlet port and configured to partially restrict a passage of air from the airflow pathway and to the ambient environment.

In another aspect of the disclosed technology, the obstruction is at least one of: a plate having one or more orifices formed therein; and a mesh screen.

In another aspect of the disclosed technology, the outlet port is configured so that a total resistance of the system to normal tidal breathing of the individual is about 1 cm H₂O/L/s or less.

In another aspect of the disclosed technology, the control unit is further configured to control the rotational speed of the impeller to produce pseudorandom noise within the passage.

In another aspect of the disclosed technology, the control unit is further configured to calculate an impedance of a respiratory system of the individual based on a measured pressure and a measured volumetric flowrate of the air within the passageway.

In another aspect of the disclosed technology, the ventilation interface includes at least one of a mouthpiece, a facemask, an endotracheal tube, a tracheal tube, a tracheostomy adapter, a tubing adapter, and a connection to a standard ventilatory interface.

In another aspect of the disclosed technology, the control unit includes a microcontroller. In another aspect of the disclosed technology, the microcontroller comprises a motor controller; and the control unit further comprises a gate driver communicatively coupled to the motor controller; and one or more field effect transistors communicatively coupled to the gate driver and configured to provide electrical current to the motor of the blower.

In another aspect of the disclosed technology, the control unit is further configured to implement a first feedback loop to control the rotational speed of the impeller to meet the set of rotational speed setpoints for the impeller.

In another aspect of the disclosed technology, the control unit is further configured to implement a second feedback loop to update the one or more rotational speed setpoints to achieve a target pressure for air within the passageway.

In another aspect of the disclosed technology, the control unit is further configured to implement the second feedback loop to at least one of: update the one or more rotational speed setpoints to a next value in the sequence of rotational speed setpoints; and compensate for changes in an actual pressure of the air within the passageway due to respiration of the individual.

In another aspect of the disclosed technology, the control unit is further configured to update the second feedback loop based a difference between the target pressure for the air within the passageway and a measurement of an actual pressure of the air within the passageway.

In another aspect of the disclosed technology, an update frequency of the first feedback loop is greater than an update frequency of the second feedback loop.

In another aspect of the disclosed technology, the update frequency of the second feedback loop is sufficient to permit the impeller to stabilize at each of the setpoints.

In another aspect of the disclosed technology, a method for evaluating the respiratory function of an individual using forced oscillation technique oscillometry includes providing a ventilation interface configured to direct breathing air to and from the individual, and providing a connecting member defining a passageway in fluid communication with the ventilation interface. The method further includes producing a substantially constant pressure offset in the passageway, and on a simultaneous basis with the production of the substantially constant pressure offset in the passage, further producing a time-varying pressure waveform in the passageway.

In another aspect of the disclosed technology, the time-varying pressure waveform is a forced oscillation technique waveform.

In another aspect of the disclosed technology, the method further includes providing a blower in fluid communication with the passageway of the connecting member, and controlling a speed of an impeller of the blower to produce the pressure offset and the time-varying pressure waveform in the passageway.

### BRIEF DESCRIPTION OF THE DRAWINGS

The following drawings are illustrative of particular embodiments of the present disclosure and therefore do not limit the scope of the present disclosure. The drawings are not to scale and are intended for use in conjunction with the explanations in the following detailed description.

FIG. 1 is a side view of a portable handheld system for evaluating the respiratory function of patients using forced oscillation technique oscillometry.

FIG. 2 is a diagrammatic illustration of the system shown in FIG. 1

FIG. 3 is a block diagram of various electrical and electronic components of the system shown in FIGS. 1 and 2.

FIG. 4 is a perspective view of a blower of the system shown in FIGS. 1-4.

FIG. 5 is an exploded view of the blower shown in FIG. 5A,

FIG. 6A depicts a single-frequency pressure waveform that can be produced by the system shown in FIGS. 1-5.

FIG. 6B depicts a multiple-frequency pressure waveform that can be produced by the system shown in FIGS. 1-5.

FIG. 6C depicts a pseudorandom noise waveform that can be produced by the system shown in FIGS. 1-5.

FIG. 7 is a flow diagram depicting a process for generating a waveform of blower speed setpoints to produce the pseudorandom noise waveform shown in FIG. 6C.

FIG. 8 is a block diagram of various electrical and electronic components of the system shown in FIGS. 1-5.

FIG. 9 is a diagrammatic illustration of the system shown in FIGS. 1-5 and 8, depicting two feedback loops implemented by the system.

FIG. 10 is a diagrammatic illustration of the system shown in FIGS. 1-5, 8, and 9 implementing a first and second feedback loop to generate an FOT waveform.

### DETAILED DESCRIPTION

The following drawings are illustrative of particular embodiments of the present disclosure and do not limit the scope of the present disclosure. The drawings are not to scale and are intended for use in conjunction with the explanations provided herein. Embodiments of the present disclosure will hereinafter be described in conjunction with the appended drawings.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. As used in this document, the term “comprising” (or “comprises”) means “including (or includes), but not limited to.” When used in this document, the terms “exemplary” and “for example” are intended to mean “by way of example” and is not intended to indicate that a particular exemplary item is preferred or required.

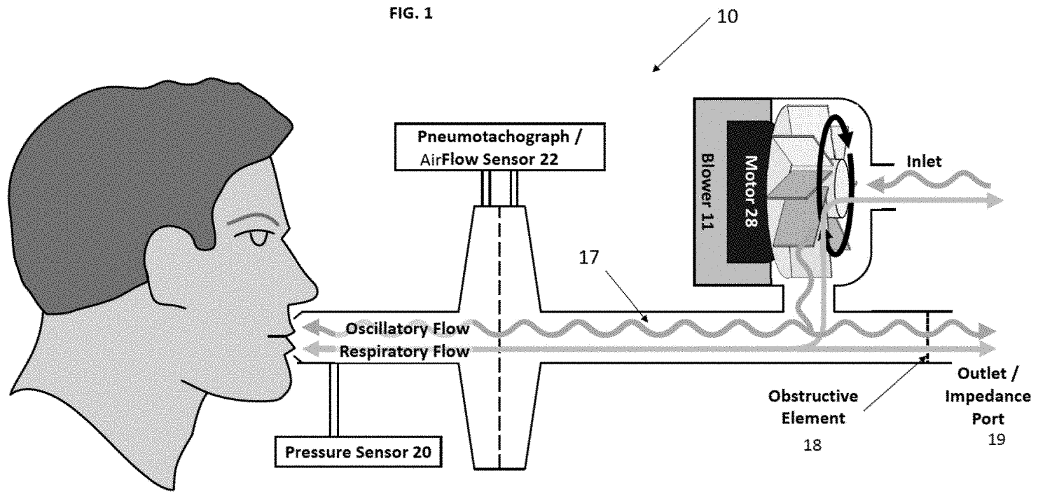

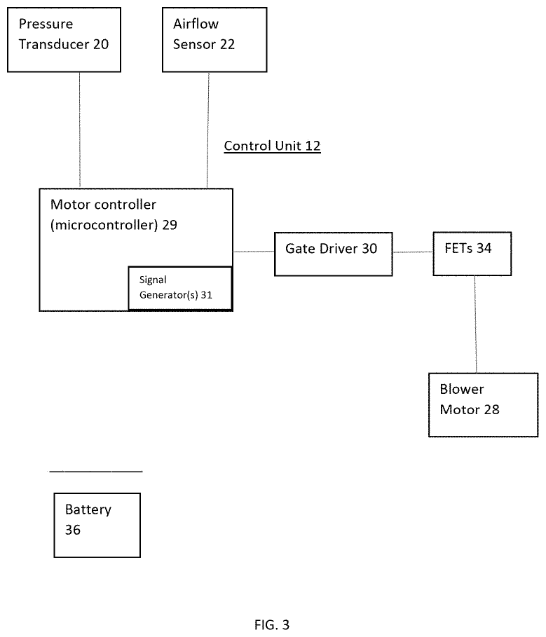

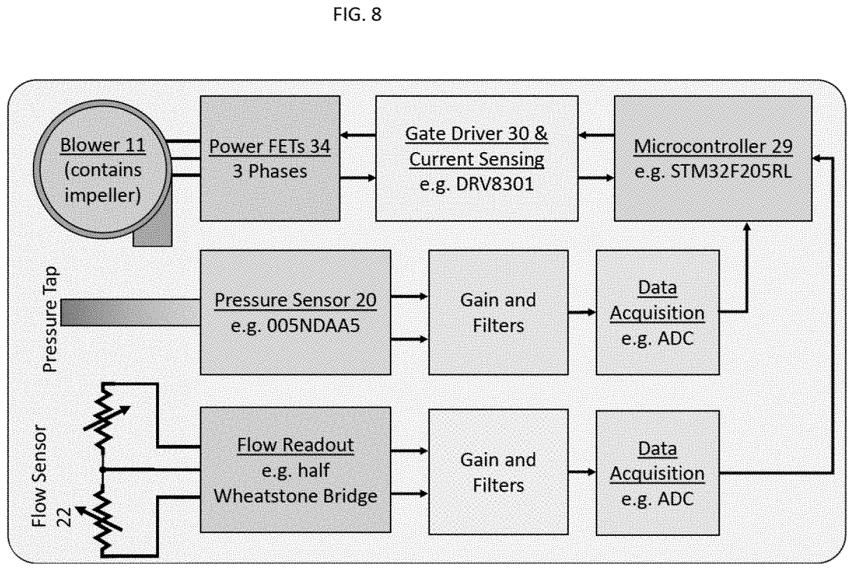

FIGS. 1-5 depict a portable handheld system 10 for evaluating respiratory function using forced oscillation technique (FOT) oscillometry. The system 10 comprises a blower 11; and a control unit 12. The blower 11 is depicted in FIGS. 1, 2, 4, and 5; the control unit 12 is depicted in FIGS. 3 and 8-10. The blower 11 acts as an air oscillation source that superimposes a sinusoidally varying pressure fluctuation on the normal tidal breathing of a patient or other individual through the large and small airways of the lungs. The control unit 12 is configured to control the speed of the blower 11 so that the blower 11 produces the desired time-varying pressure profile, or FOT pressure waveform.

In general, an FOT pressure waveform needs to have at least one frequency, and needs to be generated from at least from one sinusoidal signal. More advanced FOT pressure waveforms, containing more than one frequency and based on multiple sinusoidal signals, may be used to evaluate respiratory system behavior within the same breath. As used herein, the term “pressure waveform” is intended to mean the pressure variation, over time, generated by the blower 11 or other apparatus capable of generating a sinusoidally-varying pressure oscillation. The waveform can be generated by the blower 11 or other apparatus based on one signal provided to the blower 11 as a single control input; or a combination or summation of two or more signals provided to the blower 11 as single control input, where each signal may be periodic or aperiodic and may provide frequency content at one or more frequencies.

The control unit 12 is further configured to process the response of the patient's lungs to the FOT pressure waveform, as indicated by measurements of the pressure and volumetric flowrate of the air being inhaled and exhaled by the patient as the FOT pressure waveform is superimposed on the patient's normal tidal breathing. Specifically, the control unit 12 determines the mechanical impedance of the patient's respiratory system based on these measurements. Knowledge of the mechanical impedance of the patient's respiratory system can assist a clinician in diagnosing and treating conditions such as asthma and COPD. As discussed below, in alternative embodiments of the system 10, the calculation of the mechanical impedance of the patient's respiratory system can be made by a control unit other than the control unit 12, located remotely from the system 10.

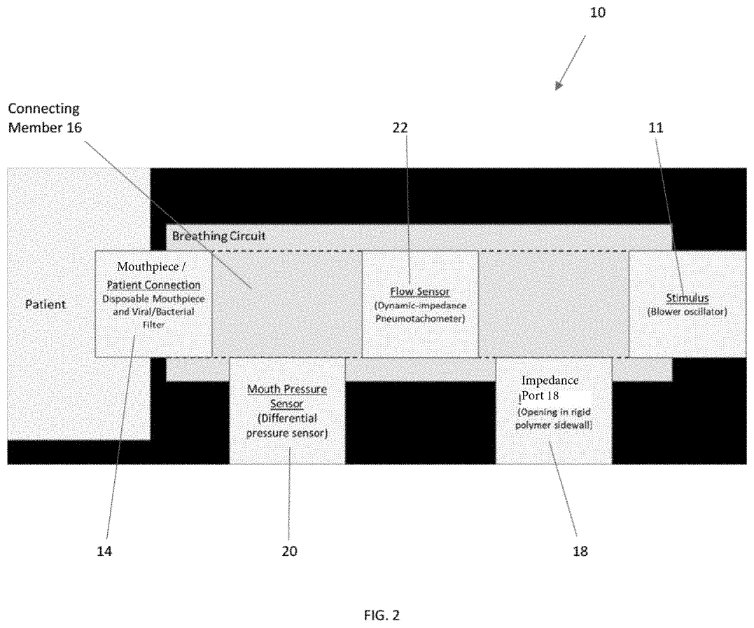

Referring to FIGS. 1 and 2, the system 10 further comprises a ventilation interface in the form of a mouthpiece 14; and a connecting member 16. The connecting member 16 is coupled physically to the blower 11 and the mouthpiece 14, and defines an internal passageway 17 that places the blower 11 in fluid communication with the mouthpiece 14. The connecting member 16 directs the FOT pressure waveform generated by the blower 11 toward the mouthpiece 104, so that the FOT pressure waveform can be superimposed on the patient's normal respiratory flow during inhalation and exhalation.

The mouthpiece 14 is placed in the patient's mouth during the diagnostic process, so that the patient inhales air transmitted to the mouthpiece 14 via the connecting member 16, and exhales into the connecting member 16 via the mouthpiece 14. The mouthpiece 14 is configured to form an airtight seal between the mouthpiece 14 and the patient's lips, so that substantially all of the air being inhaled and exhaled through the patient's mouth passes through the mouthpiece 14 and into the connecting member 16. (During the diagnostic process, the patient's nasal breathing typically will be blocked so that substantially all of the air being inhaled and exhaled by the patient passes through the mouthpiece 14 and the connecting member 16.) The mouthpiece 14 can be equipped with a viral/bacterial filter (not shown).

The ventilation interface can be any type of device suitable for allowing the patient to exhale and inhale to and from the connecting member 16, and alternative embodiments of the system 10 can include a ventilation interface other than the mouthpiece 14. For example, the ventilation interface can be a facemask, an endotracheal tube, a tracheal tube, a tracheostomy adapter, a tubing adapter, etc. As another example, the ventilation interface can be a connection to a standard ventilatory interface based on the ISO 5637 standard or other standards. The connecting member 16 can be formed from a rigid polymeric material. The internal passageway 17 within the connecting member 16 can have, for example, a circular, oval, or rectangular cross section. The passageway 17 can be configured so that the airflow within the passageway 17 remains laminar during normal operation of the system 10, and dead-space and flow obstructions within the passageway 17 are minimal or non-existent. Alternative embodiments of the connecting member 16 can have shapes, sizes, relative proportions, etc. other than those of the connecting member 16, and can be formed from semi-rigid and non-rigid materials. Also, the connecting member 16 can be unitarily formed with, or otherwise made a part of the ventilation interface or the blower 11, in other alternative embodiments.

The connecting member 16 includes an impedance port or outlet port 18, as shown in FIGS. 1 and 2. The outlet port 18 provides a pathway between the internal passageway 17 within the connecting member 16, and the ambient environment around the system 10. The pathway provided by the outlet port 18 permits the patient to breathe through the connecting member 16 and the mouthpiece 14. The outlet port 18 is sized, located, and otherwise configured so that the patient can breathe normally, while minimizing dissipation of the FOT pressure waveform generated by the blower 11.

The outlet port 18 is designed and configured as an impedance port. The term “impedance port,” as used herein, is distinct from an “inertance port.” Inertance ports are commonly used in loudspeaker-based FOT systems as low-pass filters to allow the passage of respiratory flow, at breathing frequencies, to the ambient environment, while allowing higher-frequency oscillatory flow from an FOT source to pass to the user. Inertance ports typically are configured as long tubes, to provide a volume of air sufficient to produce a desired inertive load.

The outlet port 18 has an impedance that is primarily resistive, i.e., more resistive than reactive. Due to its resistance, the outlet port 18 functions as an attenuating element for the oscillatory and respiratory flows within the system 10, without acting as a low-pass filter. The use of a blower, such as the blower 11, as a source of oscillatory flow helps to ensure that a sufficient level of oscillatory flow can delivered to the user without the use of a low-pass filter. In setting a desired maximum air pressure delivered to the user, as measured by a pressure sensor 20 of the system 10, the operational settings of the blower 11 can be adjusted to compensate for oscillatory flow that may be lost through the outlet port 18.

High inertance is not a required feature for the impedance port, i.e., the outlet port 18. Thus, the outlet port 18 can be substantially shorter than the tube used in a typical inertance port. For example, the length of the outlet port 18 can be as low as 0 to 3 inches, which can help to promote handheld form factors for the system 10. An optimal or otherwise desired impedance for a particular application can be achieved by tailoring the diameter of the outlet port 18, and/or placing an obstruction or obstructive element 19 in the outlet port 18, to vary the impedance of the system 10. The obstructive element 19 is visible in FIG. 1

It is desirable to reduce the work required by a user to breathe through the system 10, by providing a low resistance in the breathing path between the user to the ambient environment. This resistance includes a series resistance due to an airflow sensor 22 of the system 10, and the viral/bacterial filter (if so equipped); and the parallel resistance of the blower 11 and the outlet port 18. The resistance of the blower 11 may be difficult to adjust once the blower 11 is fabricated; the resistance of the outlet port 18, however, can be adjusted more easily. Thus, the impedance of the outlet port 18 can be set so that the total resistance of the system 10 to the patient's normal tidal breathing is low, e.g., less than about 1 cm H₂O/L/s.

The impedance of the impedance port, i.e., the outlet port 18, can be set by positioning the obstructive element 19 within the outlet port 18, to restrict the passage of air to the ambient environment by way of the airflow pathway within the system 10. For example, the obstructive element 19 can be a mesh in the form of woven nylon or stainless steel screens available, for example, from Component Supply Company of Sparta, Tennessee; Tex Tech Industries, Inc., of Kernersville, N.C.; and Saati S.p.A., of Appiano Gentile, Italy. Alternatively, the obstructive element 19 can be an orifice plate with one or more small, fixed-diameter openings, positioned in the airflow pathway and available, for example, from O'Keefe Controls Co., of Monroe, Connecticut; Pfeiffer Vacuum Inc., of Nashua, N.H.; Werner Solken.

The pressure sensor 20 and the airflow sensor 22 are communicatively coupled to the control unit 12. The pressure sensor 20 and the airflow sensor 22 are depicted in FIGS. 1-3. The pressure sensor 20 and the airflow sensor 22 are in fluid communication with the internal passageway 17 within the connecting member 16, so that the pressure sensor 20 and the airflow sensor 22 can sense the respective pressure and volumetric flowrate of the air being inhaled and exhaled by the patient. More specifically, the pressure sensor 20 and the airflow sensor 22 can measure changes in the pressure and volumetric flowrate of the airflow in response to the FOT pressure waveform introduced into the airflow by the blower 11.

The pressure sensor 20 is a differential pressure sensor. The pressure sensor 20 can be mounted on the connecting member 16. Alternatively, the pressure sensor 20 can be connected to the connecting member 16 by tubing or piping as shown in FIG. 1. The opening in the connecting member 16 that facilitates fluid communication between the pressure sensor 20 and the internal passageway 17 of the connecting member 16 is located proximate the end of the connecting member 16 that adjoins the mouthpiece 14, as can be seen in FIGS. 1 and 2. The pressure sensor 20 can be any type of differential pressure sensor suitable for use within the range of pressures present within the connecting member 16 during normal operation of the system 10.

The airflow sensor 22 can be a dynamic-impedance pneumotachometer, with a heated wire to prevent condensation from forming on the pneumotach screen. Other suitable types of airflow sensors can be used in alternative embodiments.

Referring to FIGS. 3-5 and 10, the blower 11 comprises a casing 24, an impeller 26 mounted for rotation within the casing 24, and a three-phase electric motor 28 configured to rotate the impeller 26 in relation to the casing 24. The blades of the impeller 26 can have a “squirrel cage” or “centrifugal” configuration. The blower 11 can be equipped with a brushless AC (BLAC) three-phase, asynchronous electric motor, such as in the AIRMAX™ P28-AC-ID blower available from Moog Inc., or the 5 kPa CPAP blower available from ASPINA.

Alternatively, the blower 11 can be equipped with a brushless DC (BLDC) electric motor, such as in the AIRMAX™ P45 series of fans and blowers, available from Moog Inc.; or the Model U71MX-024KX-4 miniature radial blower, available from Micronel AG. Other types of blowers from these and other manufacturers can be used in the alternative.

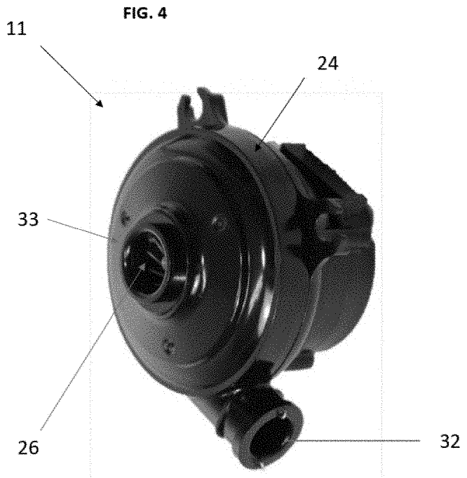

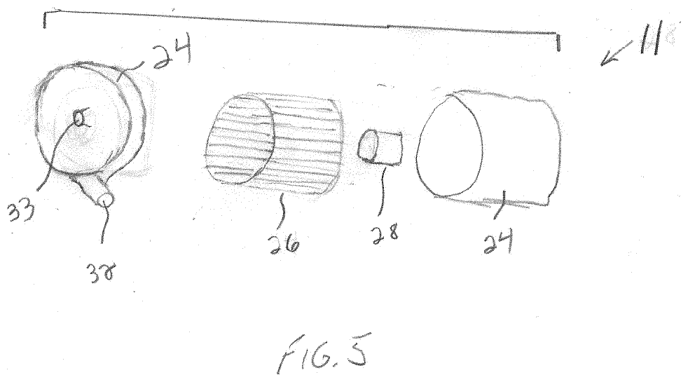

Referring to FIGS. 4 and 5, the “centrifugal” blower 11 is configured to draw ambient air from outside of the casing 24 of the blower 11, through an inlet port 32 oriented perpendicular to the casing 24. An outlet port 33 of the blower 11 is positioned within the center of the rotating impeller 26 of the blower 11. The centrifugal impeller 26 has a drum shape, and comprises a plurality of blades positioned around the outer circumference of the impeller 26. The air from the inlet port 32 is transported through the impeller 26, to the outlet port 33, and into the passageway 17 of the connecting member 16. The centrifugal blower 11 uses kinetic energy to increase or decrease the velocity and pressure of the air passing through the blower 11, thus differentiating it from a positive displacement fan or blower in an axial configuration, which uses mechanical energy to physically move air from the inlet to the outlet. The dimensions of the impeller 26 are selected to tightly match the dimensions of the adjacent internal surfaces the casing 24, to facilitate the efficient movement of air through the blower 11 and the transfer of air velocity and pressure into the passageway 17.

Details of the bower 11 are provided for illustrative purposes only. Alternative embodiments of the system 10 can be equipped with blowers having configurations other than that of the blower 11.

Referring to FIGS. 3 and 8-10, the control unit 12 comprises a microcontroller in the form of a motor controller IC, or motor controller 29 communicatively coupled to the pressure transducer 20. The control unit 12 also comprises a gate driver 30 communicatively coupled to the motor controller 29; and high and low-side field effect transistors (FETs) 34, such as but not limited to MOSFETs, communicatively coupled to the gate driver 30. The FETs 34 can provide current to the three-phase motor 28.

The gate driver 30 can be, for example, a model DRV8301 or DRV8302 gate driver available from Texas Instruments Incorporated. The gate driver 30 is configured to receive digital inputs from the motor controller 29, and to provide outputs to the FETs 34. In response, the FETs 34 control each phase of the 3-phase motor 28 by varying the current to each phase, so as to cause the impeller 26 to rotate at a rotational-speed setpoint determined by the motor controller 29.

The gate driver 30 implements a first feedback loop, discussed below, that facilitates adjustment of the rotational speed of the impeller 26 to the setpoints determined by the motor controller 29, using the current amplifiers of the gate driver 30, and the FETs 34. The rotational speed of the impeller 26 can be monitored using the back-electromotive force, or back-EMF, of the motor 28 as monitored on any or all of the 3 phases of the motor by the gate driver 30, or in alternative embodiments, by a separate IC using a differential current amplifier. The gate driver 30 can have thermal and current sensing capabilities, to help prevent damage to the motor 28 and other components of the system 10.

The motor controller 29 continuously receives pressure data from the pressure sensor 20 and airflow data from the airflow sensor 22, and implements a second feedback loop, discussed below, to update the rotational speed setpoints for the impeller 26 to obtain a target pressure for the airflow within the connecting member 16.

The control unit 12 also can include provisions to protect the electronic components of the system 10 from back-EMF, using generally known techniques.

The control unit 12 further includes a battery 36 to power the motor 28, the controller 12, and the other electronic components of the system 10. The battery 36 is depicted in FIG. 3. The battery 36 can be, for example, a lithium polymer battery. Alternative embodiments can be configured to be powered by standard 120-volt, 60 Hz household current in lieu of, or in addition to the battery 36.

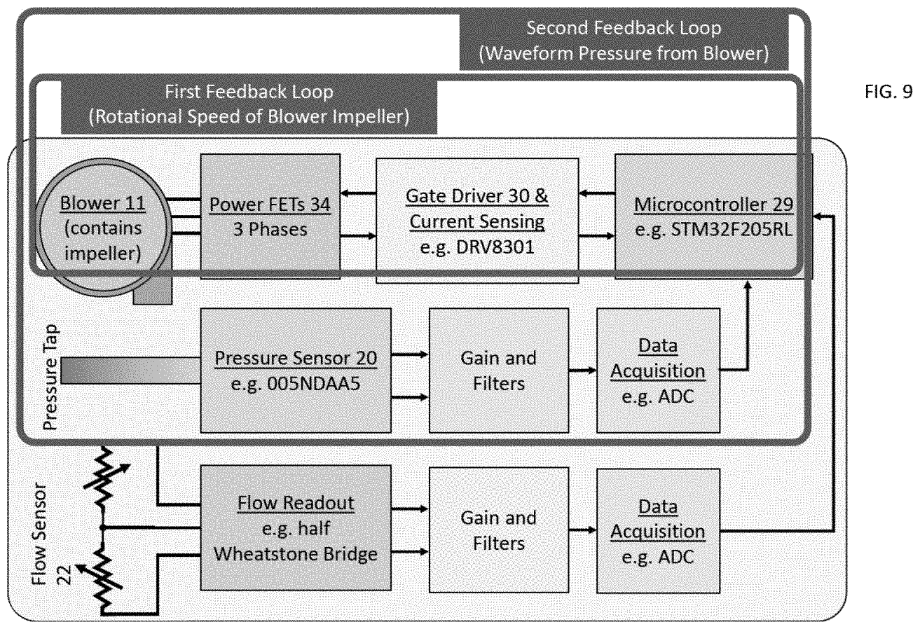

The blower 11 is controlled so that it superimposes a sinusoidally-varying pressure waveform on the normal tidal breathing of the patient. The blower 11 is controlled and stabilized using a first feedback loop implemented by the system 10 and depicted diagrammatically in FIG. 9. The first feedback loop controls the blower 11 to produce FOT pressure waveforms with maximum pressure amplitudes typically ranging, for example, from about 0.1 cm H₂O to about 2 cm H₂O; and offset pressures typically ranging, for example, from about 0.5 cm H₂O to about 40 cm H₂O.

The first feedback loop controls the rotational speed of the impeller 26 to meet a set of rotational speed setpoints using, for example, a proportional-integral-derivative (PID) control algorithm. The update frequency of the first feedback loop is relatively fast, e.g., less than about 1 millisecond (ms).

The control unit 12 can be further configured to implement a second feedback loop, also depicted in FIG. 9. The second feedback loop updates the rotational speed setpoints to obtain a targeted pressure for the airflow being inhaled and exhaled by the patient, as measured by the pressure sensor 20. The second feedback loop is optional, i.e., second feedback loop can be implemented on a continuous basis, on a periodic or intermittent basis, or not all. When the second feedback loop is not being implemented, the rotational speed setpoints can be set to either pre-calibrated or uncalibrated values to allow a user to decrease the pressure waveform during FOT testing.

The update frequency of the second feedback loop is slower than the update frequency of the first feedback loop. For example, the updated frequency of the second control loop can be about every 10 ms to 20 ms.

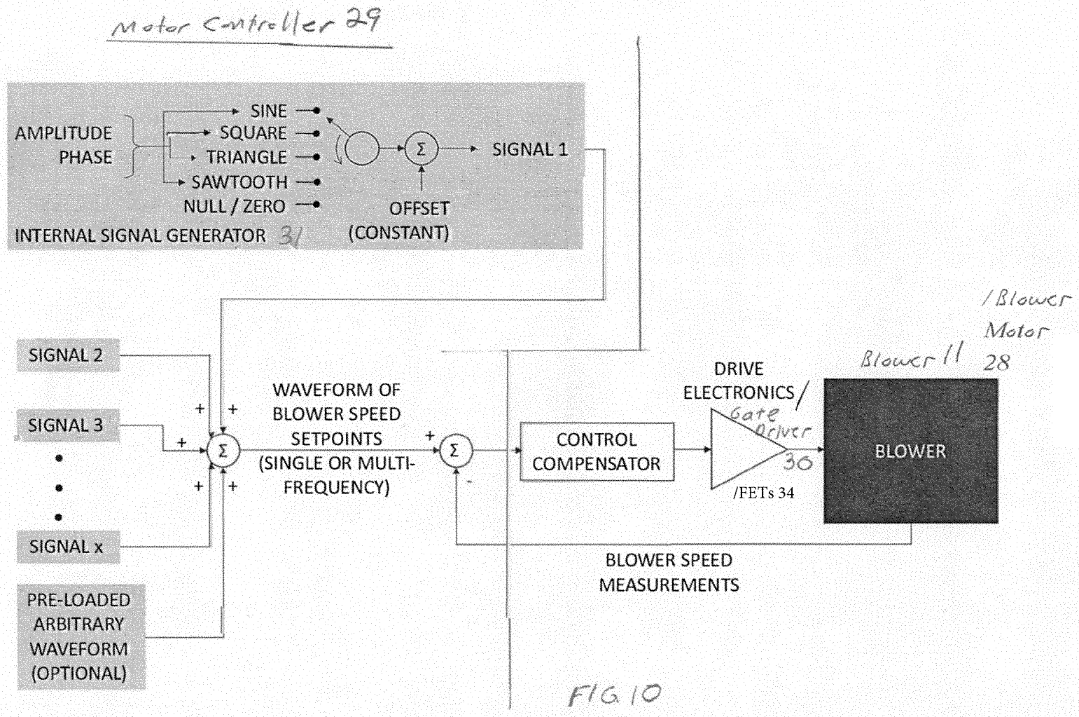

The targeted air pressure is continuously updated by the motor controller 29, based on the desired sinusoidal pressure fluctuation that is to be superimposed on the normal tidal breathing of the patient. The air pressure developed by the blower 11 approximately follows the square of the speed of the blower 11 (as reflected by the speed of the impeller 26). Thus, a particular pressure waveform can be stored in the memory of the motor controller 29 as a set of motor speed setpoints for the blower 11. Alternatively, the motor speed setpoints can be calculated on demand by one or more internal signal generators 31 within the motor controller 29, as depicted in FIG. 10.

The internal signal generator(s) 31 can generate a variety of periodic signals corresponding to the set of motor speed setpoints needed to produce a particular pressure waveform. These periodic signals can have, for example, square waveforms, sine waveforms, triangle waveforms, sawtooth waveforms, etc. Parameters of the signals, such as frequency and amplitude, can be adjusted to compensate for frequency-variant behavior of the blower 11 and its drive electronics, i.e., the gate driver 30, to generate frequency content of consistent amplitudes across all frequencies of interest. As also can be seen in FIG. 10, the internal signal generator 31 can impose a substantially constant, i.e., static or non-time-variant, offset on the pressure waveform produced by the blower 11, which can help ensure stability of the blower 11.

The internal signal generator 31 can be used for tuning coefficients or parameters of the first or second feedback loop as implemented by the gate driver 30, FETs 34, or the motor controller 29. To perform this tuning, the internal signal generator 31 can be set to generate a sequential set of signals with varying frequency components, amplitudes, offsets, or signal shapes, and then the measured pressure and flow data can be used to evaluate the overall response of the system 10. Tuning the feedback loop or loops in this way can enable more rapid transitions between specific pressure levels, improve performance across frequencies, and decrease the energy lost during electronic braking of the blower motor.

The motor controller 29 is configured to update the rotational speed setpoint based on the difference between the target pressure, and the actual pressure of the air within the connecting member 16 as measured by the pressure sensor 20, as depicted in FIG. 10. Initial motor speed estimates are supplied from the motor controller 29 using a pre-characterized pressure vs. motor speed table based on an assumption of no-load or known-load conditions. After the first feedback loop quickly drives the motor 28 to the desired setpoint, the second feedback loop can either update the motor speed setpoint to the next value in the sinusoidal waveform, or adjust the motor speed setpoints in the present and/or future waveforms to compensate for loading due to the patient's respiratory system. The adjustment of the motor speed setpoints is highly desirable to FOT, where sinusoidal amplitude pressure maxima in the range of 1 cm H₂O to 4 cm H₂O typically are required to help ensure signal integrity of flow, pressure, and ultimately transpulmonary impedance data.

The update or adjustment of the setpoints is performed at a relatively slow rate, to allow the impeller 26 to stabilize at each setpoint. Once the setpoint is updated or adjusted, the motor controller 29, implementing the first feedback loop, adjusts the rotational speed of the impeller 26 via the gate driver 30 and the FETs 34, to maintain the rotational speed of the impeller 26 at the updated setpoint.

The slower update frequency for the second feedback loop is necessary to allow the impeller 26 to stabilize at each targeted rotational speed, before the targeted speed is again adjusted to produce the desired sinusoidal pressure fluctuation on the patient's tidal breathing.

As such, the rotational speed setpoints represent a variety of thresholds to be maintained individually, using the first feedback loop, so that the blower 11 produces a pressure waveform having the desired characteristics. This approach is different from that used in conventional FOT systems comprising a speaker or a vibrating mesh, where a static pressure cannot be applied, so a constantly moving surface produces the pressure waveform; and in which there is no rotating mass, and no corresponding need to account for the moment of inertia of a rotating mass as it accelerates and decelerates, as in the system 10.

While conventional loudspeaker-based and piston-based FOT systems provide positive and negative pressure variations relative to the ambient pressure, the blower-based system 10 can be configured to provide a substantially constant offset, in relation to the ambient pressure, in the air being supplied by the blower 11. The offset is added as a substantially constant value to each point in the sinusoidal FOT pressure waveform that is to be superimposed on the normal tidal breathing of the patient. The addition of a substantially constant offset to the FOT pressure waveform can provide a small bias flow to prevent patient rebreathing of stagnant air, and/or to compensate for local ambient pressure which may see significant site-to-site variation depending on altitude, weather, temperature, etc. Also, the pressure offset helps to ensure that a positive air pressure, i.e., an air pressure above ambient, is maintained in the passage 17 of the connecting member 16, which in turn can help to minimize or substantially eliminate the potential for insufflation of the patient caused by low, or negative air pressure and airflow in the passageway 17.

Also, the pressure offset imposed on the air being supplied by the blower 11 permits the blower 11 to be controlled within the tolerances required to produce the relatively small sinusoidal variations air pressure needed for FOT oscillometry. In particular, the Applicants have found that superimposing the FOT pressure oscillations on top of a low-amplitude, substantially constant pressure offset can prevent the speed of the impeller 26 from decaying to a zero or near-zero level from which it would be difficult or impossible to accelerate sufficiently to produce the required FOT pressure oscillations. Also, the pressure offset helps to improve the signal to noise ratios in the air pressure and airflow measurements ultimately used in calculating the impedance of the patient's respiratory system.

The system 10 can be configured to produce the conventional FOT pressure waveforms discussed in the following paragraphs. These particular waveforms are disclosed for illustrative purposes only. The system 10 can be configured to produce other types of waveforms, including waveforms simultaneously comprising more than one signal. For example, the internal signal generator 31 can support the generation of two or more simultaneous signals, such as sine signals, square signals, sawtooth signals, triangle signals, etc., with the parameters of each signal capable of being set independently of the other signal. The individual signals are summed together into one combined signal, or control input, of time-varying blower speed setpoints. Optionally, the waveform may be an arbitrary or baseline waveform downloaded to the system 10 as a time-series of blower speed setpoint values, and the waveform may or may not repeat over time. A software-settable playback rate allows the effective frequency of any of the signals to be adjusted. Each signal contained in the control input optionally may compensate for frequency-variant behavior of the blower 11 and its drive electronics, i.e., the gate driver 30, to generate frequency content of consistent amplitudes across all frequencies of interest.

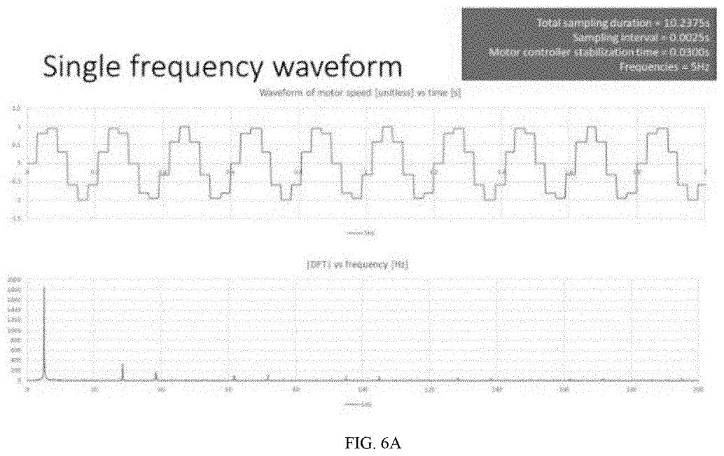

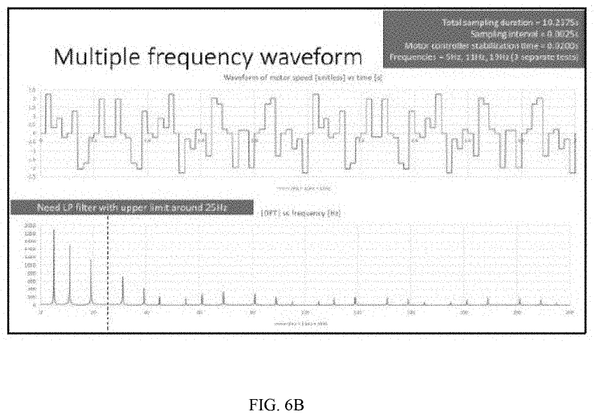

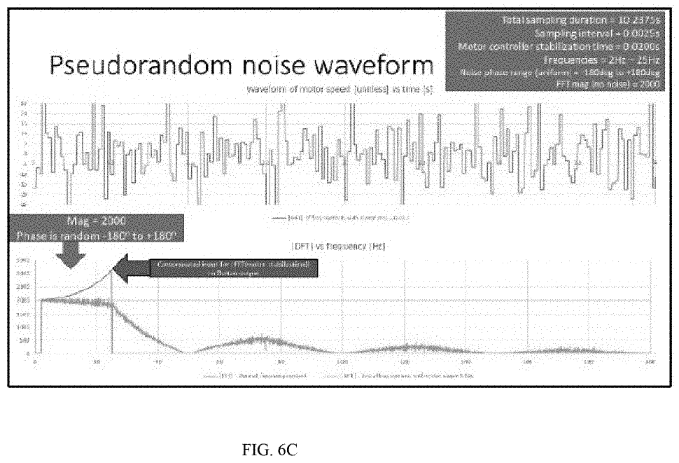

FIG. 6A depicts a conventional single-frequency FOT pressure waveform that can be produced by the system 10. In this particular example, the frequency of the waveform is 5 Hz. FIG. 6B depicts a conventional multiple-frequency FOT pressure waveform that can be produced by the system 10. In this particular example, the frequencies of the waveform are 5 Hz, 11 Hz, and 19 Hz.

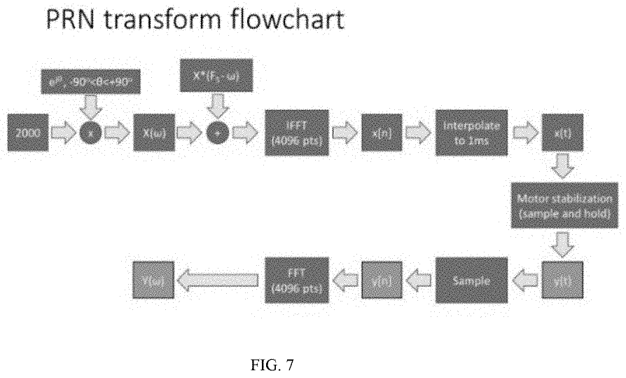

FIG. 6C depicts a pseudorandom noise that can be produced by the system 10. Pseudorandom noise enables the removal of the large pressure spike that otherwise could occur as the blower 11 is started at the beginning of diagnostic FOT process. The pressure spike can make it difficult for the blower 11 to start, and can be uncomfortable to the user. The process for generating a waveform of blower setpoints to produce a pseudorandom noise waveform is shown in the flowchart depicted in FIG. 7.

Signal to noise ratio is an important consideration in FOT oscillometry. The blower stabilization time implemented by the system 10 represents a sinc filter that is applied across all frequencies generated by the blower 11. For the single or multiple frequency waveforms, each frequency of interest optionally may be scaled to increase the signal to noise ratio; for the pseudorandom noise waveform, however, a more exact approach may be advantageous.

To compensate for the impact of stabilization of the blower 11 on the pseudorandom noise waveform, the magnitude of the input X may be scaled by 1/(the sinc function of the motor) in order to generate a flat band in the frequency domain. This can help ensure a consistent signal-to-noise ratio across the frequency range of interest, assuming that the noise is additive white gaussian noise. For example, the frequency variation in the pseudorandom waveform depicted in FIG. 6C is substantially flat in the range of about 2 Hz to about 25 Hz.

The pressure and airflow values captured by the respective pressure sensor 20 and airflow sensors 22 are communicated to the control unit 12 as the FOT pressure waveform is superimposed on the tidal breathing of the patient. The control unit 12 is configured to filter the pressure and airflow measurements to identify the pressure and flow fluctuations responsive to the FOT pressure waveform, and to extract and distinguish those fluctuations from the pressure and airflow associated with the patient's tidal breathing, using conventional techniques known generally among those skilled in the art of FOT oscillometry. The control unit 12 then calculates the impedance of the patient's respiratory system based on the pressure and flow fluctuations responsive to the FOT pressure waveform, using conventional techniques generally known in the art as algorithms for measurement of transpulmonary impedance. Such techniques may include the RLS algorithm (Recursive Least Squares to minimize the squared Equation Error, sometimes called the EE algorithm), ACOE algorithm (adjustable compensator for output error of least-squares), 2SLS algorithm (two-stage least-squares), or MLR algorithm (multiple linear regression). The time domain approaches are generally based on the Equation of Motion for a transpulmonary system that relates pressure, flow, and impedance. The frequency domain approaches involve the DFT (discrete Fourier transform), the FFT (fast Fourier transform), and IQ/PQ (in-phase or quadrature decompositions) for measuring the pressure-to-flow relationship within a narrow frequency window.

The calculated impedance of the patient's respiratory system, and the underlying pressure and airflow data, can be stored in a memory of the control unit 12, and/or can be downloaded or transmitted immediately, or at a later time.

In alternative embodiments, the pressure and airflow measurements can be transmitted a control unit remote from the system 10, such as a smartphone, a desktop or notebook computer, a server, a mainframe, etc.; and the above-noted processing of the data to yield the impedance of the patient's respiratory system can be performed by the remote control unit,

The system 10 applies pressure oscillations on top of a very low pressure offset generated by the blower 11; and the blower 11, the outlet port 18, and the patient fluidly communicate by way the connecting member 16 and the mouthpiece 14. The constant flow rate can facilitate a reduction in the size of the blower 11 and a reduction in the dead space within the airflow pathway by continuously refreshing the airflow through the parallel path of the impedance port 18 and the blower 11.

The features and functions described above, as well as alternatives, may be combined into many other different systems or applications. Various alternatives, modifications, variations or improvements may be made by those skilled in the art, each of which is also intended to be encompassed by the disclosed embodiments.

We claim:

1. A system for evaluating the respiratory function of an individual using forced oscillation technique oscillometry, comprising:
a blower comprising a casing, an impeller mounted for rotation within the casing, and a motor configured to, during operation, rotate the impeller;
a ventilation interface;
a connecting member defining a passageway in fluid communication with the blower and the ventilation interface; and
a control unit communicatively coupled to the motor and configured to control a rotational speed of the impeller to meet a set of rotational speed setpoints for the impeller so that the blower produces a time-varying pressure waveform in the passageway, the time-varying pressure waveform including a sinusoidally-varying pressure fluctuation to be superimposed on a respiratory flow of the patient by way of the ventilation interface, and an offset pressure selected to maintain a positive air pressure in the passageway during operation of the system.

2. The system of claim 1, wherein:
the control unit is further configured to control the rotational speed of the impeller to meet the set of rotational speed setpoints by generating a control input based on a desired air pressure with the passageway, and a known relationship between the rotational speed of the impeller and an air pressure produced by the blower; and
the motor is configured so that the motor varies the rotational speed of the impeller in response to the control input.

3. The system of claim 2, wherein the control input is a single-frequency signal.

4. The system of claim 2, wherein the control input is a multi-frequency signal.

5. The system of claim 2, wherein the controller is further configured to generate the control input by combining at least a first and a second signal.

6. The system of claim 5, wherein an amplitude, a phase, and a waveform of the first signal are different than a respective amplitude, phase, and waveform of the second signal.

7. The system of claim 1, wherein the offset pressure is about 0.5 cm H₂O to about 40 cm H₂O.

8. The system of claim 1, wherein the offset pressure is substantially constant.

9. The system of claim 1, wherein a maximum pressure amplitude of the time varying pressure waveform is about 0.1 cm H₂O to about 2 cm H₂O.

10. The system of claim 1, further comprising an outlet port in fluid communication with the passageway in the connecting member, and an ambient environment around the system.

11. The system of claim 10, wherein the outlet port has a length of about zero to about three inches.

12. The system of claim 10, wherein the passageway and the outlet port form an airflow pathway between the ventilation interface and the ambient environment around the system; and
the system the further comprises an obstruction located within the outlet port and configured to partially restrict a passage of air from the airflow pathway and to the ambient environment.

13. The system of claim 12, wherein the obstruction is at least one of: a plate having one or more orifices formed therein; and a mesh screen.

14. The system of claim 10, wherein the outlet port is configured so that a total resistance of the system to normal tidal breathing of the individual is about 1 cm H₂O/L/s or less.

15. The system of claim 1, wherein the control unit is further configured to control the rotational speed of the impeller to produce pseudorandom noise within the passage.

16. The system of claim 1, wherein the control unit is further configured to calculate an impedance of a respiratory system of the individual based on a measured pressure and a measured volumetric flowrate of the air within the passageway.

17. The system of claim 1, wherein the ventilation interface comprises at least one of a mouthpiece, a facemask, an endotracheal tube, a tracheal tube, a tracheostomy adapter, a tubing adapter, and a connection to a standard ventilatory interface.

18. The system of claim 1, wherein the control unit comprises a microcontroller.

19. The system of claim 18, wherein:
the microcontroller comprises a motor controller; and
the control unit further comprises a gate driver communicatively coupled to the motor controller; and one or more field effect transistors communicatively coupled to the gate driver and configured to provide electrical current to the motor of the blower.

20. The system of claim 1, wherein the control unit is further configured to implement a first feedback loop to control the rotational speed of the impeller to meet the set of rotational speed setpoints for the impeller.

21. The system of claim 20, wherein the control unit is further configured to implement a second feedback loop to update the one or more rotational speed setpoints to achieve a target pressure for air within the passageway.

22. The system of claim 21, wherein the control unit is further configured to implement the second feedback loop to at least one of: update the one or more rotational speed setpoints to a next value in the sequence of rotational speed setpoints; and compensate for changes in an actual pressure of the air within the passageway due to respiration of the individual.

23. The system of claim 21, wherein the control unit is further configured to update the second feedback loop based a difference between the target pressure for the air within the passageway and a measurement of an actual pressure of the air within the passageway.

24. The system of claim 23, wherein an update frequency of the first feedback loop is greater than an update frequency of the second feedback loop.

25. The system of claim 24, wherein the update frequency of the second feedback loop is sufficient to permit the impeller to stabilize at each of the setpoints.

26. A method for evaluating the respiratory function of an individual using forced oscillation technique oscillometry, comprising:
providing a ventilation interface configured to direct breathing air to and from the individual;
providing a connecting member defining a passageway in fluid communication with the ventilation interface; and
producing a substantially constant pressure offset in the passageway; and
on a simultaneous basis with the production of the substantially constant pressure offset in the passage, further producing a time-varying pressure waveform in the passageway.

27. The method of claim 26, wherein the time-varying pressure waveform is a forced oscillation technique waveform.

28. The method of claim 26, further comprising:
providing a blower in fluid communication with the passageway of the connecting member; and
controlling a speed of an impeller of the blower to produce the pressure offset and the time-varying pressure waveform in the passageway.
